(19) United States
(12) Patent Application Publication
(10) Pub. No.: US 2010/0291565 A1
(43) Pub. Date: Nov. 18, 2010

(54) Detection of Methylation in Nucleic Acid Sequences

(76) Inventors: Michael James Wilkinson, Ceredigion (GB); Carlos Marcelino Rodriguez Lopez, Aberystwyth (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/669,545

(22) PCT Filed: Jul. 18, 2008
(86) PCT No.: PCT/GB2008/002487

§ 371 Date: Apr. 13, 2010

(30) Foreign Application Priority Data
Jul. 19, 2007 (GB) 0714058.5

(51) Int. Cl. C12Q 1/68; G01N 33/50

(52) U.S. Cl. 435/6; 436/94

(57) ABSTRACT

The present invention provides a method for detecting and/or quantifying the presence of, and relative abundance of, methylated nucleic acid bases within double-stranded nucleic acid by i) contacting a double-stranded nucleic acid sample with an intercalating fluorescent dye when bound to the nucleic acid sample fluoresces when exposed to light of a wavelength capable of causing the dye to fluoresce; 2) altering the hybridisation conditions of the solution containing the double-stranded nucleic acid-dye complex such that dissociation of the two strands of the said nucleic acid-dye complex occurs at a rate that permits progressive release of the dye 3) and monitoring the difference in fluorescence, and uses thereof.

The present invention relates to a method for detecting methylation on nucleic acid sequences, research tools therefore, and uses thereof. In particular, the invention relates to a method for detecting methylation on DNA derived from biological samples, research tools therefore, and uses thereof.

DNA methylation appears to be widespread across many distinct eukaryotic phyla such as plants, mammals, birds and the invertebrates such as nematodes (Finnegan et al., 1998; Suzuki S, et al., 2007; Gupta S et al., 2006; delGaudio R et al., 1997). In plants, DNA methylation has been linked with gene silencing, for example, in Arabidopsis thaliana hyper-methylated alleles of genes SUP, PAI, and AG (Jacobsen and Meyerowitz, 1997; Melquist et al., 1999; Jacobsen at al., 2000) and in Linaria, LCYC (Cubas et al., 1999). However, hypo-methylation of alleles AP3 and FWA in Arabidopsis thaliana displayed ectopic expression (Kinoshita et al., 2004). Several reports suggest a role for DNA methylation in the regulation of plant development and cell type (Finnegan at al., 2000)

DNA methylation is also known to play a role in the epigenetic control of the expression of genes in mammals, including humans, and may be linked with certain tissue types. Determining the degree of methylation of particular genomic DNA target regions of interest is useful in research fields. In humans and other mammals methyl cytosine is found almost exclusively in cytosine-guanine (CpG) dinucleotides. Genomic DNA methylation plays an important role in gene regulation. Among the earliest and most common genetic alterations observed in human malignancies such as cancers, is the aberrant methylation of so-called CpG islands, particularly CpG islands located within the 5′ regulatory regions of genes, causing alternations in the expression of such genes.

There is considerable research interest in the role of DNA methylation inter alia in the areas of embryogenesis, cellular differentiation, and transcriptional regulation.

The methods of detecting differences in methylation patterns on nucleic acids described in the prior art appear to be complex relative to the method of the invention described herein.

WO 2004/065625 describes the use of intercalating, modified oligonucleotides, that is to say intercalating nucleic acids, as probes for targeting nucleic acid, typically DNA. The method uses such oligonucleotides because they possess additional components that alter the stability of their binding, leading to more stable nucleic acid: intercalating oligonucleotide couplets that can be used as a basis for detecting methylation on cytosine bases in the test nucleic acid strand. However, this requires that the strand test is first modified to accept methyl groups, for example by use of bi-sulphidising techniques. Such techniques are not utilized in the present invention.

US2003/0224434 describes a method for detecting nucleic acid sequence variants in nucleic acids in a sample that inter alia relies on the use of labeled primers that comprise a fluorescent label. Labeled primers are not utilized in the present invention.

WO 2000/046398 describes a method for detecting methylated nucleic acids wherein the method relies on the contacting of an oligonucleotide sequence, that is to say a labeled oligonucleotide sequence, wherein a portion of it is labeled with fluorophore moiety and a second portion of it is labeled with a quencher moiety that is capable of quenching the fluorophore moiety when the fluorophore moiety is in sufficiently close proximity to it. Labeled oligonucleotides as described in WO 2000/046398 are not utilized in the present invention.

U.S. Pat. No. 7,153,671 (equivalent of WO 2001/007647) describes a method for the relative quantification of the methylation of cytosine bases in DNA samples wherein genomic DNA of the sample is reacted with a reagent, such as bisulphite ion, to generate differential reactions of 5-methyl cytosine and cytosine thus enabling an alteration in base pairing behaviour which is exploited in the invention of U.S. Pat. No. 7,153,671. The method by which the base pairing behaviour is generated as described in U.S. Pat. No. 7,153,671 is not utilised in the present invention.

WO 02/034942 describes methods of measuring nucleic acid methylation where such methods include a bisulfite conversion step which converts unmethylated cytosine to uracil but does not similarly convert methylated cytosine.

Hsiung et al. (1976) Can. J. Biochem. 54 pp 1047-54, described the effect of treating DNA with chemicals that generate a form of DNA that is not present in nature (with methylation occurring in the deoxyguanosine residues instead of the cytosine residues). Hsiung et al. then measured only the level of fluorescence generated by ethidium bromide after chemical modification of DNA is reduced but did not measure the initial level of methylation in a sample of DNA.

It is an object of the present invention to provide a simpler method than those described in the prior art for detecting methylation on nucleic acid in a sample. In particular there is provided a method of detecting methylation of naturally occurring methylated nucleic acids. This and other objects of the invention will become apparent from the foregoing description and examples.

According to the present invention there is provided a method of detecting methylation of nucleic acid in a sample by
i) contacting said nucleic acid with a chemical agent that is capable of generating a detectable signal when contacted therewith;
ii) denaturing the modified nucleic acid formed in i) to a single stranded state in a steadily controlled manner;
iii) monitoring changes in the detectable signal from the chemical agent in contact with nucleic acid during the denaturation step ii); and
iv) determining the level of methylation of nucleic acid by comparing the signal profile of test nucleic acid with that generated from control samples of nucleic acid having a known methylation pattern.

The method of the invention as described above is a high resolution melt (HRM) method requiring the denaturation (melting) of double stranded DNA.

Preferably the methylation being detected is a naturally occurring methylation of a nucleic acid e.g. the detection of methyl-cytosine.

The chemical agent used may occupy an intercalating position with the nucleic acid and may be of any type such as a modified nucleotide base comprising a modified nucleotide base comprising a fluorescing agent such as a fluorescent dye, such as one selected from ethidium bromide, LC Green, SYBR Green I, YO-PRO-1, BEBO, EVA GREEN and SYTO9; a chromophore, such as a chromophore dye, such as one selected from Cy3, Pyr(10)PC; or a chromophore dye pair suitable for use in FRET analysis (Kainmueller and Bannwarth, 2006) such as the dye pair TOTO 1 and TOTO 3.

Fluorescent dyes as contemplated for use in methods of the invention are ones which are able to combine (for example, via intercalation) with the nucleic acid of the sample to form complexes that are capable of giving out a detectable signal, for example, upon exposure to particular wavelengths of light. Suitable fluorescent dyes for use in methods of the invention include ethidium bromide, LC Green, SYBR Green I, YO-PRO-1, BEBO and SYTO9 as outlined herein. SYTO9 is particularly preferred for use in methods of the invention since SYTO9 produces highly reproducible DNA melting curves over a broad range of dye concentrations. The high melting curve reproducibility of SYTO9 indicates that it can be readily incorporated into a closed tube analysis by DNA melting curve at a broad range of target DNA concentrations. These features improve reproducibility of DNA melting curve and so make SYTO9 useful in a diagnostic context (Monis et al., 2005).

The method may also include the optional step of contacting the nucleic acid and the reaction mixture with a fluorophore quenching agent. Quenching agents decrease the fluorescence intensity of a given substance. A variety of processes can result in quenching, such as excited state reactions, energy transfer, complex-formation and collisional quenching. The addition of a quenching agent may assist in the process of discrimination between bound and non-bound fluorophores.

The denaturing of nucleic acid to which the chemical agent is contacted, for example, via intercalation, can be brought about by altering the stringency of the hybridisation conditions in a controlled manner, such as by increasing temperature in an incremental manner as outlined herein. It is well known that at low stringency conditions, for example, at temperatures below the melting temperature (Tₘ) of target nucleic acid, such as deoxyribonucleic acid (DNA), that it generally maintains a double stranded structure in which an intercalating chemical agent embedded in it, such as described herein will emit a signal: a chemical agent that is an intercalating dye such as those described herein, will fluoresce. When the nucleic acid begins to dissociate with increase in stringency conditions, such as a gradual, controlled increase in temperature or a gradual controlled increase in chemical denaturant concentration such as an increase in SSC, urea or MgCl₂ concentration in the reaction mix (thereby altering the binding stability of double-stranded nucleic acid at a given temperature, typically causing a gradual unravelling of a helical structure), the chemical agent, for example an intercalating agent, is released from the nucleic acid as it unravels, which gives rise to a gradual decrease in fluorescence associated with the nucleic acid. The alteration in fluorescence between such single stranded nucleic acid such as DNA, can be discriminated as stringency conditions are increased in a controlled manner, leading to an alteration in the binding behaviour of the chemical agent. Preferably the chemical agent is an intercalating agent. For example, by constructing Tₘ curves with increase in stringency conditions, for example incremental increases in temperature, differences in the binding ability of chemical agents such as intercalating agents give rise to different observable patterns of detectable fluorescence. Methylated and hemi-methylated nucleic acid strands not only have different melt temperatures but also give rise to distinct melting curve shapes that are different from one another and from that of unmethylated DNA over certain temperature ranges. The preferred temperature range will depend on the length and sequence of the target nucleic acid but would ordinarily fall within the range 30-90° C., preferably from 40-90° C., and more preferably from 60-90° C. for fragments of length less than 500 bases, preferably from 10-400 bases, more preferably from 20-300 bases and most preferably from 20-200 bases. Furthermore, the proportion of methylated and hemi-methylated nucleic acid strands also determines the shape of the melting curve and Tₘ. By comparing the methylation profile generated by a test DNA sample to an unmethylated standard, known DNA sample, for example, a standard sample derived by PCR or nucleic acid synthesis, and/or a standardised methylated DNA sample, for example, one derived by chemical nucleic acid synthesis or a DNA sequence isolated from a biological sample of known base sequence and methylation pattern, nucleic acid of the same base sequence provides a reference scale against which the proportion of methylation can be determined. There are several methods that may be deployed to infer the relative degree of methylation in a biological sample when compared with standard controls, including visual curve comparisons to known synthesised mixtures (methylated and unmethylated reference mixes), by multivariate analysis of the curves, or by comparison of the fluorescence levels under increased stringency conditions, such as fluorescence at specified temperatures within the curves relative to reference mixtures.

The skilled addressee will appreciate that where the difference in Tₘs between a given methylated nucleic acid and its corresponding non-methylated nucleic acid fragment is not known several control reactions may be performed in parallel with the test sample as contemplated herein. Reference controls should include components such as the corresponding nucleic acid sequence in unmethylated form, the corresponding nucleic acid sequence in which all double stranded fragments comprise methylated bases, and a mixture or mixtures containing set, different proportions of unmethylated and methylated nucleic acid. Preferably the nucleic acid that is used as reference control is DNA that may be synthetic or purified and of known sequence. Oligonucleotides used in the invention as controls can be synthesized by a number of approaches known in the art (Beaucage, 2001). The oligonucleotides of the invention are typically synthesized on an automated DNA synthesizer, e.g. by Sigma Aldrich using standard chemistries and following the manufacturer's instructions.

Preferably, multi-valent cations (such as CaCl₂ or MgCl₂) and/or small proteins such as spermidine, or peptides containing alternating lysine bases are used to stabilize differentially methylated and non-methylated nucleic acids such as DNA strands and DNA oligonucleotides. Methylated DNA is able to be preferentially stabilized at close to physiological salt concentrations, whereas higher salt concentrations, for example >10 mM, such as provided for in the examples, are required to achieve a stabilising effect on unmethylated fragments (Behe & Felsenfield, 1981). Both multivalent cations and spermidine stabilize DNA by shielding the more closely spaced phosphates in the Z-DNA backbone (Kim et al., 2006). Equally, peptides containing appropriately spaced lysine amino acid residues also help to stabilize its structure by co-operative shielding of phosphate backbones of Z-DNA (Kim et al., 2006). It is well established that Mg⁺⁺ has a powerful stabilizing influence on methylated nucleic acid structures, such as DNA structure. In a particularly preferred embodiment of the invention, the denaturation reaction is performed in the presence of a sufficient concentration of Mg⁺⁺ ions that is capable of facilitating the discrimination between methylated and non-methylated DNA fragments on the basis of DNA melt curve profiles.

It is preferable that reaction mixtures containing methylated and non-methylated nucleic acid fragments, such as methylated and non-methylated DNA fragments also contain a salt such as MgCl₂ (or other divalent cation such as Ca⁺⁺, e.g. CaCl₂), in sufficient concentration, for example in the range from 0.01 mM to 10 mM, to increase the stability of the methylated fragments. Examples of the MgCl₂ concentration that may be used to stabilize nucleic acid in the method of the invention include 0.5, 1.5, 2.5, 3, 5 and 10 mM. A suitable concentration of MgCl₂ is 2.5 mM.

Similarly, the effect of different spermidine concentrations on the denaturing profiles of different nucleic acid fragments, such as methylated and non-methylated DNA fragments, has an effect on the stability of the methylated fragments. A suitable spermidine concentration range for use in the method of the present invention is from 0.1 μM to 2 μM. Examples of the spermidine concentration that may be used to stabilize methylated nucleic acid in the method of the invention include 0.1, 0.2, 0.5, 1, 1.5 and 2 μM. Preferably the concentration of the spermidine is 1.5 mM. Therefore certain conditions (such as those explain above) in the dilution where DNA is suspended increase the differences in Tₘs of methylated and non-methylated fragments.

The method of the invention may also include additional optional steps of amplification of the nucleic acid being analysed. Amplification of the nucleic acid can be conducted using any DNA amplification method including thermocyclic amplification methods such as the polymerase chain reaction (PCR). Such methods are well known in the art and are described in textbooks such as Sambrook and Russell, Molecular Cloning:a Laboratory Manual. Volumes 1, 2 and 3 (2001, 3^{RD} Ed).

The use of a methyltransferase in an amplification reaction is applicable to any method of detecting methylation of DNA. Amplification of the DNA, for example by PCR results in DNA strands which are only hemi-methylated i.e. only one strand (the parent strand) is methylated. Inclusion of a methyltransferase into the reaction mixture ensures that the methylation from the parent strand is then translated onto the replicated strand. As such, use of a methyltransferase enzyme allows the original nucleic acid sample to be amplified and retain the methylation pattern of the parent nucleic acid sample. This amplification with retained methylation pattern improves the sensitivity of methylation detection assays.

Methods of detecting methylation in DNA include but are not limited to High resolution melts (HRM), bisulfate conversion and genome wide epigenetic analysis.

Preferably the methyltransferase enzyme is used in a method of improving the detection of methylation of a nucleic acid sample comprising by contacting the nucleic acid sample with the methyltransferase. Most preferably, the nucleic acid sample has been amplified prior to contacting with the methyl transferase enzyme.

The nucleic acid under investigation may optionally be contacted with a methyltransferase enzyme. Where an amplified nucleic acid is contacted with the methyltransferase, the methyltransferase restores any methylation lost during the amplification reaction.

As a further aspect of the invention is also provided a kit for detecting methylation of nucleic acid, that comprises: (a) a chemical agent as herein defined; (b) at least one control nucleic acid sample of known methylation status and optionally (c) a chemical denaturant as herein defined.

The invention will now be illustrated in the accompanying Figures and Examples. It is to be understood that the teaching of the examples is not to be construed as limiting the scope of the invention in any way.

### FIGURE LEGENDS

FIG. 1. Principal Component Analysis diagram based on the analysis of the fluorescence readings obtained from the melting curves obtained from a normalized denaturation profile of two mixtures containing entirely methylated (seven sites) and a mix containing 97.5% methylated DNA and 2.5% unmethylated DNA, and containing MgCl₂ at 3 mM or spermidine at 1.5 μM. (▴) Represents three replicates of a mix containing 97.5% methylated DNA and 1.5 μM spermidine, () represents three replicates of a mix containing 100% methylated DNA fragments and 1.5 μM spermidine, (♦) represents three replicates of a mix containing 97.5% methylated DNA fragments and 2.5 mM MgCl₂, (▪) represents three replicates of a mix containing 100% methylated DNA fragments and 2.5 mM MgCl₂.

FIG. 2 a, b. and c. Shows normalized denaturation profiles of various DNA mixtures comprizing methylated and non-methylated target DNA sequences at different proportions at three different DNA concentrations. As illustrated in the figures, purple traces represent eight replicates of methylated DNA, orange traces represent eight replicates of a mix containing 50% methylated DNA (7 sites) and 50% unmethylated DNA, purple traces represents eight replicates of a mix containing 0% methylated DNA fragments.

2a) Shows normalized denaturation profiles of various mixtures containing methylated and non-methylated target DNA sequences at different proportions (as above) at a DNA concentration of 0.02 mM;

2b) Shows normalized denaturation profiles of various mixtures containing methylated and non-methylated target DNA sequences at different proportions at a DNA concentration of 0.002 mM;

2c) Shows normalized denaturation profiles of various mixtures containing methylated and non-methylated target DNA sequences at different proportions at a DNA concentration of 0.2 μM.

FIG. 3. Variation in the strength of fluorescence signal generated by three dyes (Syto 09, Syber green and Eva Green) when applied to identical templates of methylated, hemi-methylated and unmethylated DNA synthesised to the same sequence of a section of the CNR gene from tomato (described below)

FIG. 4. High resolution melt (HRM) analysis of DNA representing a fragment of the CNR gene of tomato with identical base sequence but in unmethylated, hemi-methylated and methylated state using Syto 09.

FIG. 5. HRM analysis of DNA representing a fragment of the CNR gene of tomato with identical base sequence but in unmethylated, hemi-methylated and methylated state (Eva green left; Syber green right).

FIG. 6. HRM profiles of de novo synthesised DNA with identical base sequence taken from the tomato CNR gene but with differing Cytosine methylation status: Samples were either unmethylated on both strands, methylated on one strand only (hemimethylated); methylated on both strands (methylated); hemimethylated but incubated with methyl transferase for 10 min at 37 C prior to HRM (Hemimethylated+enzyme) or methylated but incubated with methyl transferase for 10 min at 37 C prior to HRM (Methylated+enzyme).

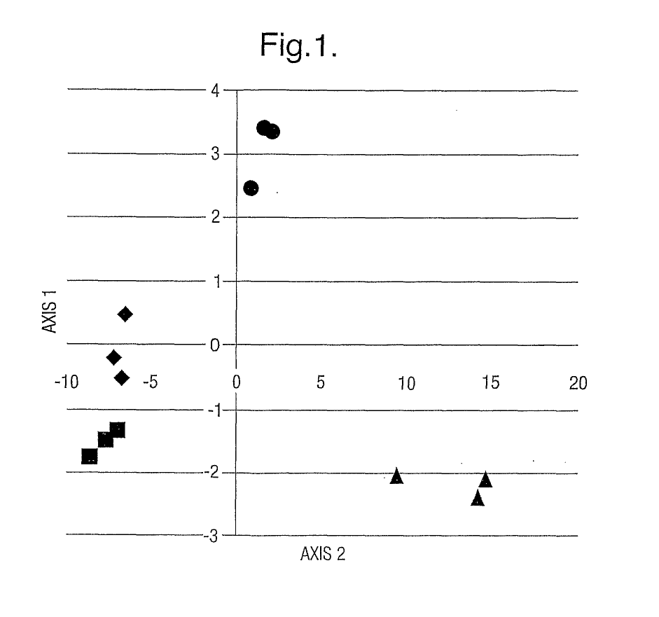

### EXAMPLES

Methods used in molecular biology that are applicable in the following Examples are known to the man skilled in the art. General tests that describe conventional molecular biology, microbiology, and recombinant DNA techniques known to the man skilled in the art, included, for example: Sambrook et al., Molecular Cloning: A Laboratory Manual, Second Edition, Cold Spring Harbor Laboratory Press, Cold Spring Harbor, N.Y. (1989); Glover ed., DNA Cloning: Practical Approach, Volumes I and II, MRL Press, Ltd., Oxford, U.K. (1985); and Ausubel, F., Brent, R., Kingston, R. E., Moore, D. D., Seidman, J. G T., Smith J. A., Struhl, K. Current protocols in molecular biology. Green Publishing Associates/Wiley Intersciences, New York.

### Example 1

### Thermal Effects on Denaturation

### Target DNA

To investigate the temperature profiles of methylated, hemimethylated, and non-methylated DNA using DNA dyes, oligonucleotides of 77 bases representing a hypothetical region of an Eukaryote and their complementary sequences were purchased from Sigma Aldrich. The oligonucleotides contained 7 CG sites which included cytosine residues that are able to be methylated. In the methylated oligonucleotides 1, 2, 3 or 7 cytosines within the 7 CG dinucleotides were replaced with 5 methyl-cytosines.

Two other fragments were also studied: 1) a 32 bases DNA fragment with base sequence from the human gene Myf3 and containing 7 CG sites which included cytosine residues that are able to be methylated. In the methylated oligonucleotides 7 cytosines within the 7 CG dinucleotides were replaced with 5 methyl-cytosines. and 2) a 85 bases fragment with base sequence from tomato Cnr gene. The oligonucleotides contained 3 CG and 1 CNG sites which included cytosine residues that are able to be methylated. In the methylated oligonucleotides 1, 2, 3, or 4 cytosines within the 4 four possible sites were replaced with 5 methyl-cytosines.
The non-methylated oligonucleotides remain unmethylated. All the purchased oligonucleotides were HPLC purified.

TABLE 1
Oligonucleotide Sequences

| | |
|---|---|
| Non-modified | 5′AGGACATGGAGTGGAACGAGGAGCTGCAGGC |
| | GTTCACGTACCCGTGTCCCTGCGGCGACCTGTT |
| | CCAGATCACGAGG3′ |
| Non-modified | 5′CCTCGTGATCTGGAACAGGTCGCCGCAGGGA |
| reverse complement | CACGGGTACGTGAACGCCTGCAGCTCCTCGTTC |
| | CACTCCATGTCCT3′ |
| Modified 7CH3 | 5′AGGACATGGAGTGGAAOGAGGAGCTGCAGGO |
| | GTTCAOGTACCOGTGTCCCTGOGGOGACCTGTT |
| | CCAGATCAOGAGG3′ |
| Reverse | 5′CCTOGTGATCTGGAACAGGTOGCOGCAGGGA |
| complement | CAOGGGTAOGTGAAOGCCTGCAGCTCCTOGTTC |
| 7CH3 | CACTCCATGTCCT3′ |
| Modified 1CH3 | 5′AGGACATGGAGTGGAAOGAGGAGCTGCAGGC |
| | GTTCACGTACCCGTGTCCCTGCGGCGACCTGTT |
| | CCAGATCACGAGG3′ |
| Modified reverse | 5′CCTCGTGATCTGGAACAGGTCGCCGCAGGGA |
| complement 1CH3 | CACGGGTACGTGAACGCCTGCAGCTCCTOGTTC |
| | CACTCCATGTCCT3′ |
| Modified 2CH3 | 5′AGGACATGGAGTGGAAOGAGGAGCTGCAGGC |
| | GTTCACGTACCOGTGTCCCTGCGGCGACCTGTT |
| | CCAGATCACGAGG3′ |
| Modified reverse | 5′CCTCGTGATCTGGAACAGGTCGCCGCAGGGA |
| complement 2CH3 | CAOGGGTACGTGAACGCCTGCAGCTCCTOGTTC |
| | CACTCCATGTCCT3′ |
| Modified 3CH3 | 5′AGGACATGGAGTGGAAOGAGGAGCTGCAGGC |
| | GTTCACGTACCOGTGTCCCTGCGGCGACCTGTT |
| | CCAGATCAOGAGG3′ |
| Myf3 | 5′GCGGCGACTCCGACGCGTCCAGCCCGCGCTC |
| | C3′ |
| Reverse complement | 5′GGAGCGCGGGCTGGACGCGTCGGAGTCGCCG |
| Myf3 | C3′ |
| Myf3 (7CH3) | 5′GOGGOGACTCOGAOGOGTCCAGCCOGOGCTC |
| | C3′ |
| Reverse complement | 5′GGAGOGOGGGCTGGAOGOGTOGGAGTOGCOG |
| Myf3 (7CH3) | C3′ |
| CNR | 5′GAATTACCCGTTGTTTCAATTGATGAAGTGT |
| | TTTAATCTGACACTTCCGGTTCGTTGTTATTCC |
| | TATACTAGATTGTTAAGTTAA3′ |
| Reverse complement | 5′TTAACTTAACAATCTAGTATAGGAATAACAA |
| CNR | CGAACCGGAAGTGTCAGATTAAAACACTTCATC |
| | AATTGAAACAACGGGTAATTC3′ |
| CNR (4CH3) | 5′GAATTACCOGTTGTTTCAATTGATGAAGTGT |
| | TTTAATOTGACACTTCOGGTTOGTTGTTATTCC |
| | TATACTAGATTGTTAAGTTAA3′ |
| Reverse complement | 5′TTAACTTAACAATCTAGTATAGGAATAACAA |
| CNR (4CH3) | OGAACOGGAAGTGTOAGATTAAAACACTTCATC |
| | AATTGAAACAAOGGGTAATTC3′ |
| CNR (3CH3) | 5′GAATTACCCGTTGTTTCAATTGATGAAGTGT |
| | TTTAATOTGACACTTCOGGTTOGTTGTTATTCC |
| | TATACTAGATTGTTAAGTTAA3′ |
| Reverse | 5′TTAACTTAACAATCTAGTATAGGAATAACAA |
| complement | OGAACOGGAAGTGTOAGATTAAAACACTTCATC |
| CNR (3CH3) | AATTGAAACAACGGGTAATTC3′ |
| CNR (2CH3) | 5′GAATTACCCGTTGTTTCAATTGATGAAGTGT |
| | TTTAATCTGACACTTCOGGTTOGTTGTTATTCC |
| | TATACTAGATTGTTAAGTTAA3′ |
| Reverse complement | 5′TTAACTTAACAATCTAGTATAGGAATAACAA |
| CNR (2CH3) | OGAACOGGAAGTGTCAGATTAAAACACTTCATC |
| | AATTGAAACAACGGGTAATTC3′ |
| CNR (1CH3) | 5′GAATTACCCGTTGTTTCAATTGATGAAGTGT |
| | TTTAATCTGACACTTCCGGTTOGTTGTTATTCC |
| | TATACTAGATTGTTAAGTTAA3′ |
| Reverse complement | 5′TTAACTTAACAATCTAGTATAGGAATAACAA |
| CNR (1CH3) | OGAACCGGAAGTGTCAGATTAAAACACTTCATC |
| | AATTGAAACAACGGGTAATTC3′ |

Where “O” stands for 5 methyl-cytosine

### Oligonucleotide Hybridization Conditions

Double stranded DNA fragments were constructed by adding oligonucleotides at equal proportions and heating them at 95° C. for five minutes and letting them cool slowly to room temperature over 15 minutes. In order to determine the effect of methylation and hemi-methylation the oligonucleotide combinations shown in Table 2 were created.

TABLE 2
Tested oligonucleotide combinations

| 5′-3′ fragment | 3′-5′ fragment |
|---|---|
| Non-modified | Non-modified reverse complement |
| Modified 7CH3 | Reverse complement 7CH3 |
| Modified 7CH3 | Non-modified reverse complement |
| Myf3 | Reverse complement Myf3 |
| Myf3 (7CH3) | Reverse complement Myf3 (7CH3) |
| Myf3 (7CH3) | Reverse complement Myf3 |
| Myf3 | Reverse complement Myf3 (7CH3) |
| CNR | Reverse complement CNR |
| CNR (4CH3) | Reverse complement CNR (4CH3) |
| CNR (4CH3) | Reverse complement CNR |
| CNR | Reverse complement CNR (4CH3) |
| CNR (3CH3) | Reverse complement CNR (3CH3) |
| CNR (2CH3) | Reverse complement CNR (2CH3) |
| CNR (1CH3) | Reverse complement CNR (1CH3) |

### Reaction Mix

Melting curve experiments were conducted by adding appropriate concentrations (such as 0.2 μM) of double stranded DNA fragments to a reaction mixture containing 0-10 mM MgCl₂ or 0.1-2.0 μM spermidine, 1.6 mM (NH₄)SO₄, 6.7 mM Tris-HCl (pH 8.8 at 25° C.) and 0.001% Tween-20. The dye was added at a final concentration of 0.5 μM.

### Thermal Denaturation Conditions

Intercalating DNA dyes are known to link to double stranded DNA at certain temperatures. The bound dyes fluoresce, indicating the occurrence of hybridization. In order to determine the effects of changing temperatures on the level of fluorescence, thermal denaturation profiles were carried out with different DNA fragments. The fluorescence was monitored during each profile by using a Rotor Gene 6000 available from Corbett Life Science, Sydney, Australia.

The profile included increasing the temperature from 50° C. to 90° C. at 0.1° C. steps with each temperature being held for 2 seconds. The fluorescence was monitored eight times at each temperature step.

The fluorescence intensity for each sample as temperature increased was plotted as a function of temperature on a linear scale from 0-100. The magnitude differences between samples in the original data could be normalized. This was performed using the software incorporated on the Corbett Rotor-Gene 6000 real-time rotary analyzer by which two linear regions are selected, one before and one after the major transition. These regions defined two lines for each curve, an upper, 100% fluorescence line and a lower, 0% fluorescence line. The melt curves from the raw data are then normalized by averaging all starting and ending fluorescence values and then forcing the end points of each sample to be the same as the average. These results are shown as normalized results. In doing this, each DNA type (methylated, hemimethylated or non-methylated) and each methylated or non-methylated DNA proportion are grouped together. Different types of samples have similar melting curve shapes but different Tₘs, without significant differences between replicates. Representative profiles are shown in TABLES 3 and 4.

TABLE 3 (a, b and c) Shows normalized fluorescence values from various mixtures containing methylated, hemimethylated, and non-methylated target DNA sequences (performed using the software incorporated on the Corbett Rotor-Gene 6000 real-time rotary analyzer by which the melt curves from the raw data are normalized by averaging all starting and ending fluorescence values and then forcing the end points of each sample to be the same as the average). 100% Methylated DNA values represent the mean for eight replicates of methylated DNA (methylated in seven positions as described on the text), Hemimethylated DNA values represent eight replicates of the same DNA sequence, where only the forward strand is methylated, reverse hemimethylated values represent eight replicates of the same DNA sequences where only the reverse strand is methylated, 0% Methylated DNA values represent eight replicates of non-methylated DNA of the same sequence (as described on the text) Between parenthesis are indicated the calculated standard error from the mean fluorescence.

3a) DNA fragments 77 bp long representing a hypothetical region of an Eukaryote

| | Normalized Fluorescence | | | |
|---|---|---|---|---|
| Temperature | | | Reverse | |
| (° C.) | 0% Methylated DNA | Hemimethylated DNA | hemimethylated DNA | 100% Methylated DNA |
| 84.40 | 22.53 (0.19) | 29.94 (0.26) | 31.97 (0.27) | 32.13 (0.23) |
| 86.00 | 6.39 (0.11) | 14.67 (0.26) | 17.62 (0.21) | 21.37 (0.16) |
| 87.00 | 2.06 (0.24) | 5.07 (0.16) | 7.06 (0.18) | 13.56 (0.13) |

3b) 32 bp long DNA fragment from the human gene Myf3

| | Normalized Fluorescence | | | |
|---|---|---|---|---|
| Temperature | | Hemimethylated | Reverse hemimethylated | |
| (° C.) | 0% Methylated DNA | DNA | DNA | 100% Methylated DNA |
| 80.54 | 37.68 (0.51) | 47.97 (0.85) | 52.37 (0.72) | 57.41 (0.30) |
| 82.54 | 21.77 (0.45) | 32.13 (0.79) | 36.55 (1.00) | 43.34 (0.46) |
| 84.54 | 8.96 (0.66) | 17.73 (0.67) | 21.10 (0.93) | 29.92 (0.60) |

3c) 85 bp long DNA fragments from tomato Cnr gene

| | Normalized Fluorescence | | | |
|---|---|---|---|---|
| Temperature | | Hemimethylated | Reverse hemimethylated | |
| (° C.) | 0% Methylated DNA | DNA | DNA | 100% Methylated DNA |
| 75.73 | 42.44 (2.00) | 51.22 (1.68) | 55.78 (0.51) | 60.32 (0.52) |
| 76.73 | 7.60 (0.54) | 13.48 (2.06) | 19.63 (0.71) | 29.60 (1.68) |
| 77.73 | 4.37 (0.05) | 4.49 (0.09) | 5.06 (0.08) | 6.71 (0.44) |

### Example 2

### Determination of the Effects of Salt Concentration and Spermidine

Multivalent cations and other molecules such as spermidine and peptides containing alternating lysines stabilize differentially methylated and non-methylated oligonucleotides. At a gross level it is know that methylated DNA is preferentially stabilized at close to physiological salt concentrations. However, higher salt concentrations (e.g. MgCl₂ or CaCl₂) are needed to achieve the same effect on non-methylated fragments (Behe & Felsenfield, 1981). Both multivalent cations and spermidine stabilize DNA by shielding the more closely spaced phosphates in the Z-DNA backbone. Equally, properly spaced lysines co-operatively shield phosphate backbones of Z-DNA helping to stabilize its structure (Kim et al., 2006). Mg⁺⁺ has a powerful stabilizing influence on the methylated DNA fragment. The hybridization reaction is carried out in the presence of a sufficient concentration of Mg⁺⁺ ions that is capable of facilitating the discrimination between methylated and non-methylated DNA fragments. The effects of MgCl₂ concentrations on denaturing profiles of the different oligonucleotides were investigated. Reaction mixtures containing methylated and non-methylated DNA fragments were subjected to 0.5, 1.5, 2.5, 3, 5 and 10 mM MgCl₂ (TABLE 5).

Similarly, the effects of different spermidine concentrations on the denaturing profiles of the different oligonucleotides were investigated. Reaction mixtures containing methylated and non-methylated DNA fragments were subjected to 0.1, 0.2, 0.5, 1.0, 1.5, and 2.0 μM (TABLE 5). Preferably the concentration of spermidine was 1.5 μM (TABLE 5).

### Effects of Salt and Spermidine on the Denaturation Profiles

Analysis of different salt concentrations with methylated and non-methylated DNA fragments indicated that the presence of MgCl₂ in the reaction mixture affects the differences between both types of fragments peaking at a MgCl₂ concentration of 3 mM. On the other hand spermidine presented an optimum at 1.5 μM (Table 5). The sensitivity of the system ranges from being able to discriminate between mixes containing 0 and 1% of methylated DNA fragments to discriminate mixes containing 97.5 and 100% of methylated DNA fragments (TABLE 6).

Table 5 Effect of MgCl₂ and spermidine on the denaturation profiles Shows fluorescence values obtained by subtracting the fluorescence of a mix containing 97.5% methylated DNA curves from the fluorescence of 100% methylated DNA curves obtained when melts were performed with different MgCl₂ (0.5, 1.5, 2.5, 3, and 10 mM) or spermidine (0.1, 0.2, 0.5, 1, 1.5, and 3 mM) concentrations in the incubating solution. The total represents the average fluorescence difference at different temperatures ranging from 82.01 to 92.01° C. taken at intervals of 0.1° C.

| | Δ Fluorescence (100%-97.5% Methylated DNA) | | | | | | | | | | | |
|---|---|---|---|---|---|---|---|---|---|---|---|---|
| Temperature | [MgCl₂] (mM) | | | | | | [Spermidine] (μM) | | | | | |
| (° C.) | 0.5 | 1.5 | 2.5 | 3 | 5 | 10 | 0.1 | 0.2 | 0.5 | 1 | 1.5 | 2 |
| 84.01 | −0.48 | −0.20 | 0.21 | 0.52 | −0.13 | −0.85 | 1.19 | 1.55 | 1.54 | 2.56 | 1.80 | 1.41 |
| 86.01 | −0.04 | 0.46 | 0.58 | 0.81 | 0.38 | −0.56 | 2.94 | 3.72 | 4.13 | 6.15 | 6.79 | 5.05 |
| 88.01 | 0.24 | 0.67 | 0.74 | 1.02 | 1.23 | 0.02 | 1.59 | 2.18 | 3.38 | 5.80 | 7.92 | 6.32 |
| Total | −0.09 | 0.19 | 0.40 | 0.61 | 0.37 | −0.41 | 1.46 | 1.89 | 2.20 | 3.59 | 3.96 | 3.09 |

### Example 3

### Determination of the Denaturation Profile

In order to construct a denaturation profiles for methylated, hemimethylated, and non-methylated DNA fragments (the first two carrying 7-methylated cytosines), the temperature was increased from 50° C. to 90° C. at 0.1° C. increments with each temperature being held for 2 seconds. The fluorescence was monitored eight times at each temperature step. This resulted in a typical inverted sigmoid curve. As the temperature was raised, the level of fluorescence decreased. This was due to the denaturation of the double stranded DNA structure to a single stranded structure leading to release of the intercalating dye which is only able to fluoresce when linked to double stranded DNA.

Fluorescence presents a maximum at 50° C. and decreases steadily until a certain temperature was reached, from where the fluorescence decreased rapidly. This temperature represented the temperature at which the double stranded DNA fragment melts. For the methylated DNA fragment the hybridization melt temperature was approximately 89° C. The hybridization melt temperature for the hemimethylated fragments depended on which strand was methylated, the fragment carrying the methylation on the modified reverse complement strand melted at approximately 87.5° C., while the fragment carrying the methylation on the forward strand presented a melting temperature of approximately 88° C. For the non-methylated DNA fragment hybridization melt temperature was approximately 86.5° C.

### Effects of Methylated DNA/Non-Methylated DNA Proportion on the Denaturation Profiles

Once the proposed methodology was proven capable of detecting differences on methylation profiles between methylated, hemimethylated and non-methylated DNA, it was tested to determine whether if it was possible to distinguish between samples containing different proportions of methylated and non-methylated DNA. This aim was achieved obtaining melt profiles for 17 different mixes of methylated/non-methylated DNA such as 0, 1, 2.5, 5, 10, 20, 35, 45, 50, 55, 60, 65, 75, 85, 90, 95, and 100% methylated DNA. As shown in table 4, the proportion of methylated DNA fragments in a mix alters its melting curve and melting temperature. Using this methodology, it is possible to distinguish between mixes with as little as 5% in their proportion of methylated DNA.

Table 4 (a, b and c) Effects of methylated DNA/non-methylated DNA proportion on the denaturation profiles shows normalized fluorescence values from various mixtures containing methylated and non-methylated target DNA sequences at different proportions. As illustrated in the Table, each tabulated value represents the mean of eight replicates of: methylated DNA; a mix containing 75% methylated DNA fragments and 25% unmethylated DNA of the same sequence; a mix containing 50% methylated DNA fragments; a mix containing 25% methylated DNA fragments; a mix containing 5% methylated DNA fragments; and unmethylated DNA fragments of the same sequence (described herein). Between parenthesis are indicated the calculated standard error from the mean fluorescence.

| | Normalized Fluorescence | | | | | |
|---|---|---|---|---|---|---|
| | 0% | 5% | 25% | 50% | 75% | 100% |
| Temperature | Methylated | Methylated | Methylated | Methylated | Methylated | Methylated |
| (° C.) | DNA | DNA | DNA | DNA | DNA | DNA |

4a) DNA fragments 77 bp long representing a hypothetical region of an Eukaryote

| | | | | | | |
|---|---|---|---|---|---|---|
| 82.52 | 63.95 (0.12) | 64.83 (0.11) | 65.70 (0.11) | 69.92 (0.08) | 70.50 (0.08) | 71.12 (0.06) |
| 85.02 | 27.75 (0.13) | 29.94 (0.14) | 32.00 (0.15) | 43.50 (0.14) | 47.22 (0.10) | 48.81 (0.07) |
| 86.02 | 13.31 (0.07) | 15.64 (0.10) | 17.86 (0.10) | 30.04 (0.16) | 34.93 (0.11) | 37.20 (0.09) |

4b) 32 bp long DNA fragment from the human gene Myf3

| | | | | | | |
|---|---|---|---|---|---|---|
| 80.54 | 38.88 (1.00) | 42.48 (0.32) | 47.95 (0.32) | 55.74 (0.33) | 59.10 (1.00) | 62.74 (0.37) |
| 82.54 | 18.39 (1.67) | 24.12 (0.27) | 31.30 (0.4) | 40.87 (1.33) | 44.79 (0.43) | 49.23 (0.44) |
| 84.54 | 6.90 (0.64) | 10.61 (0.37) | 17.10 (0.25) | 29.96 (1.06) | 31.51 (0.45) | 37.10 (0.53) |

4c) 85 bp long DNA fragments from tomato Cnr gene

| | | | | | | |
|---|---|---|---|---|---|---|
| 75.73 | 54.87 (0.55) | 58.36 (0.30) | 60.64 (0.60) | 62.28 (0.75) | 64.59 (0.65) | 67.23 (0.67) |
| 76.73 | 19.26 (0.80) | 25.94 (0.37) | 31.10 (0.19) | 35.11 (0.81) | 39.62 (0.94) | 44.37 (0.93) |
| 77.73 | 6.43 (35) | 7.53 (0.14) | 9.69 (0.07) | 12.29 (0.45) | 15.65 (1.03) | 20.63 (0.37) |

TABLE 6 Shows fluorescence values obtained from normalized denaturation profile of two mixtures containing entirely methylated (seven sites) and a mix containing 97.5% methylated DNA and 2.5% unmethylated DNA, and containing MgCl₂ at 2.5 mM, or spermidine at 1.5 μM. Between parenthesis are indicated the calculated standard error from the mean fluorescence.

| | Normalized Fluorescence | | | |
|---|---|---|---|---|
| | [MgCl₂] (3 mM) | | [Spermidine] (1.5 μM) | |
| | 97.5% Methylated | 100% Methylated | 97.5% Methylated | 100% Mehtylated |
| Temperature (° C.) | DNA | DNA | DNA | DNA |
| 83.51 | 53.98 (0.18) | 54.21 (0.07) | 49.48 (0.38) | 50.50 (0.23) |
| 85.51 | 38.97 (0.23) | 39.60 (0.15) | 28.36 (0.57) | 31.53 (0.27) |
| 87.51 | 19.02 (0.16) | 20.47 (0.11) | 8.15 (0.33) | 11.54 (0.08) |

### Example 4

### Effect of Fragment Concentration

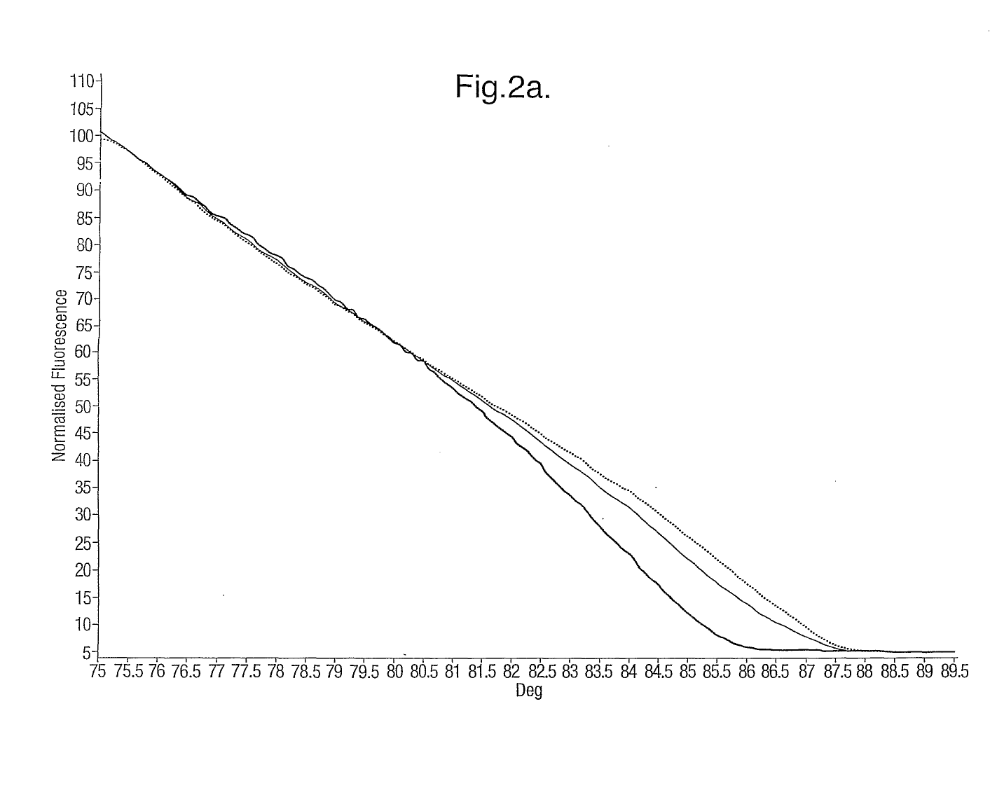

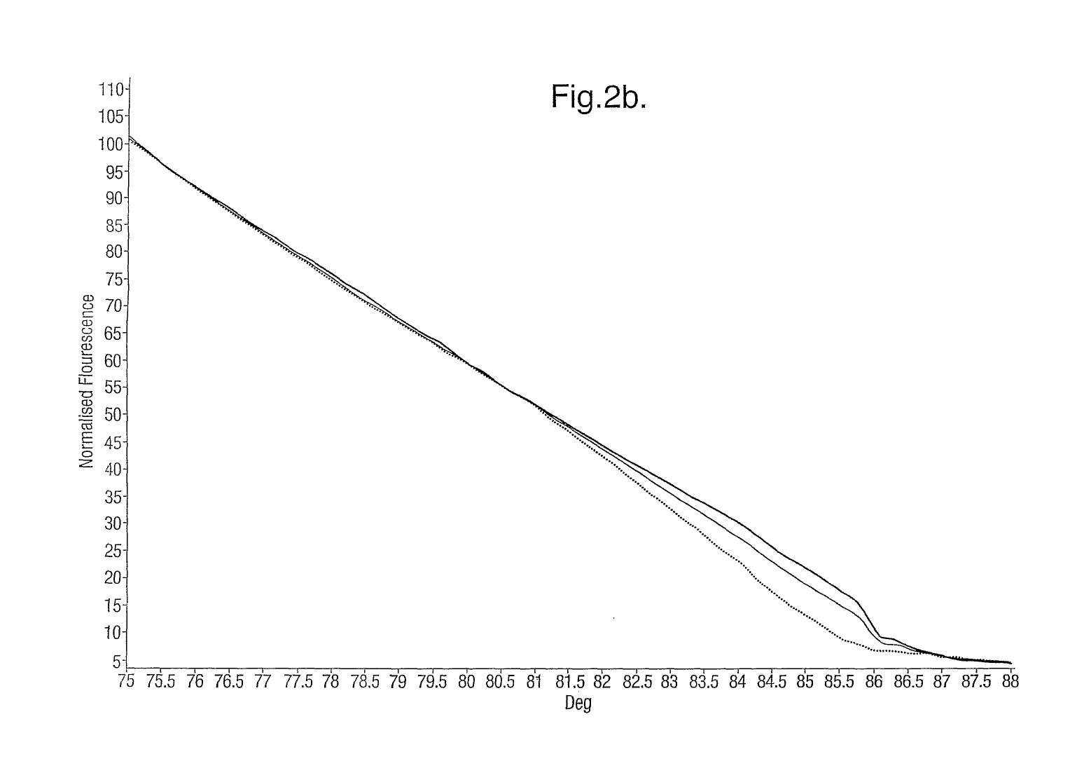

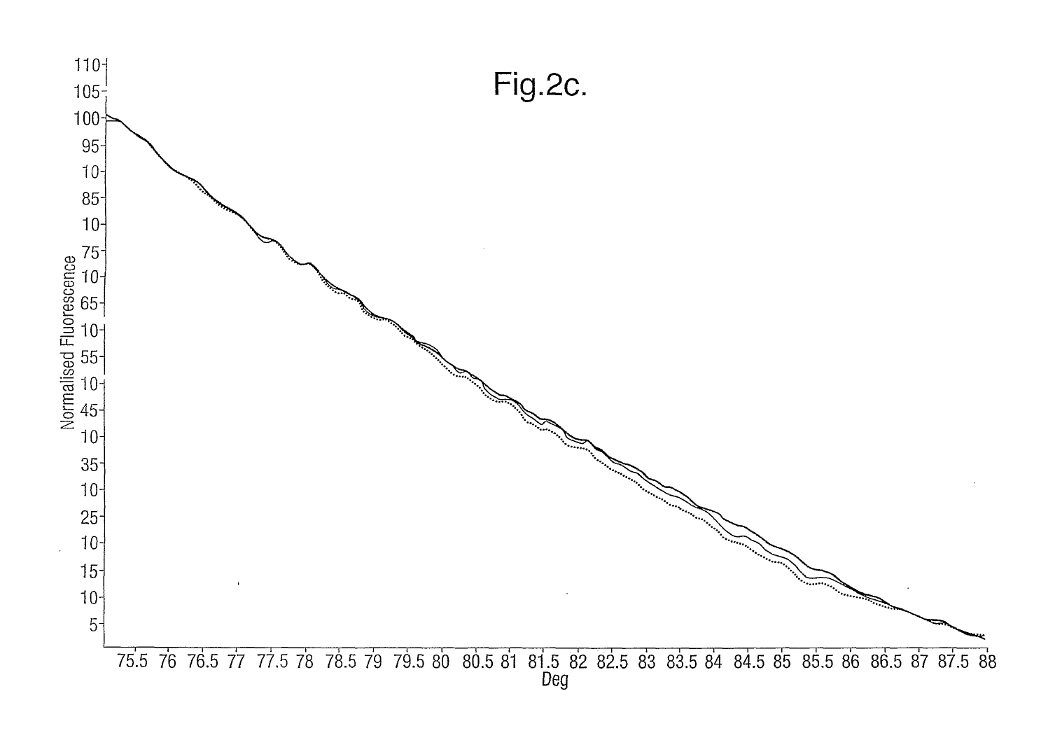

In order to determine the minimum amounts of DNA fragments and of intercalating dye needed for an optimal detection of the denaturation profiles, serial dilutions of the DNA fragments were carried out within the range from 0.2 μM to 0.2 nM. Three different methylated/non-methylated DNA mixes including 0, 50, and 100% methylated DNA. Distinct melting temperatures or melting curves between the three different mixes were obtained when using dilutions up to 0.2 nM (See FIGS. 2a, 2b, and 2c). Below that threshold, fluorescence signal is not high enough above background levels to detect the melting point and simply decreases as temperature increases in linear relation with it. The target DNA fragment was present in a concentration of at least 0.2 nM.

Table 7 Effect of DNA fragment concentration on denaturation profiles Shows normalized denaturation profiles of various DNA mixtures comprizing methylated and non-methylated DNA fragments 77 bp long representing a hypothetical region of an Eukaryote genome at different proportions at three different DNA concentrations. The table contains data from eight replicates of methylated DNA, eight replicates of a mix containing 50% methylated DNA (7 sites) and 50% unmethylated DNA, and eight replicates of a mix containing 0% methylated DNA fragments.
Column a) Shows normalized denaturation profiles of various mixtures containing methylated and non-methylated target DNA sequences at different proportions (as above) at a DNA concentration of 0.02 μM;
Column b) Shows normalized denaturation profiles of various mixtures containing methylated and non-methylated target DNA sequences at different proportions at a DNA concentration of 0.002 μM;
Column c) Shows normalized denaturation profiles of various mixtures containing methylated and non-methylated target DNA sequences at different proportions at a DNA concentration of 0.2 nM.

| | DNA concentration | | | | | | | | |
|---|---|---|---|---|---|---|---|---|---|
| | 0.02 μM | | | 0.002 μM | | | 0.2 nM | | |
| | 0% | 50% | 100% | 0% | 50% | 100% | 0% | 50% | 100% |
| Temp | Methylated | Methylated | Methylated | Methylated | Methylated | Methylated | Methylated | Methylated | Methylated |
| (° C.) | DNA | DNA | DNA | DNA | DNA | DNA | DNA | DNA | DNA |
| 81.00 | 54.18 (0.38) | 55.53 (0.14) | 55.96 (0.09) | 51.72 (0.22) | 51.78 (0.04) | 52.17 (0.08) | 45.98 (0.28) | 47.01 (0.13) | 47.16 (0.25) |
| 83.00 | 33.96 (0.51) | 39.69 (0.17) | 41.92 (0.11) | 32.85 (0.14) | 35.78 (0.06) | 37.54 (0.08) | 29.65 (0.14) | 31.53 (0.20) | 32.30 (0.20) |
| 85.00 | 11.85 (0.40) | 21.69 (0.22) | 25.94 (0.15) | 13.26 (0.07) | 19.07 (0.11) | 22.04 (0.08) | 16.08 (0.16) | 17.50 (0.11) | 18.87 (0.11) |

### Example 5

### Sensitivity of Detection of Methylated Cytosines

The method described above can readily detect methylation of the mentioned target DNA when all 7 cytosines with in the CpG dinucleotides are methylated (See Table 1). To determine the minimum number of cytosine residues that are needed to be methylated in order to alter the melt temperature of a DNA fragment compared to a same sample completely unmethylated compared to a completely unmethylated similar sample, DNA fragments with variable numbers of methylated cytosines residues were melted simultaneously with non-methylated DNA fragments. As shown on table 1, multiple different DNA fragments were examined. Modifications ranged from nil to three cytosine residues methylated. As illustrated on TABLE 8 the sensitivity of the system is able to discriminate between sample differing in only one methylated cystosine.

Table 8 Effects of number of methylated cytosines on the denaturation profiles Shows normalized fluorescence values from various mixtures containing methylated, carrying 1, 2 or 3 methylated cytosines, and non-methylated 85 by long DNA fragments from tomato Cnr gene, as illustrated in the Table 1. Each tabulated value represents the mean of eight replicates of each type of DNA fragment. Between parenthesis are indicated the calculated standard error from the mean fluorescence.

| | Normalized Fluorescence | | | |
|---|---|---|---|---|
| Temperature | 0 methylated | 1 methylated | 2 methylated | 3 methylated |
| (° C.) | cytosines | cytosine | cytosines | cytosines |
| 76.03 | 39.57 (1.06) | 51.76 (0.77) | 56.93 (0.92) | 62.66 (0.70) |
| 76.53 | 20.21 (0.80) | 34.52 (0.98) | 42.30 (1.12) | 51.68 (1.07) |
| 77.03 | 9.77 (0.42) | 17.69 (0.73) | 25.02 (1.06) | 38.16 (1.30) |

### Example 6

### Enhancing Sensitivity of Detection

Scope for exploitation of the method using genomic DNA isolated from organisms requires a sufficient concentration of the target DNA to enable melt profiles from methylated and non-methylated DNA to be distinguished. In some instances, this could be achieved through the isolation of large quantities of template DNA from an individual (especially for species with large body mass such as many vertebrate mammals or for perennial plants species with multiple ramets representing individual genotypes). Where this is not possible, or where the quantity of template approaches the current limits of detection, differentiation can be achieved through improved sensitivity of detection or through amplification of the target DNA.

### Improved Sensitivity of Signal Detection

There are numerous means by which signal detection can be improved using existing technology. One approach would be to exploit the innate variability between the fluorescent intercalating dyes for detecting DNA melts.

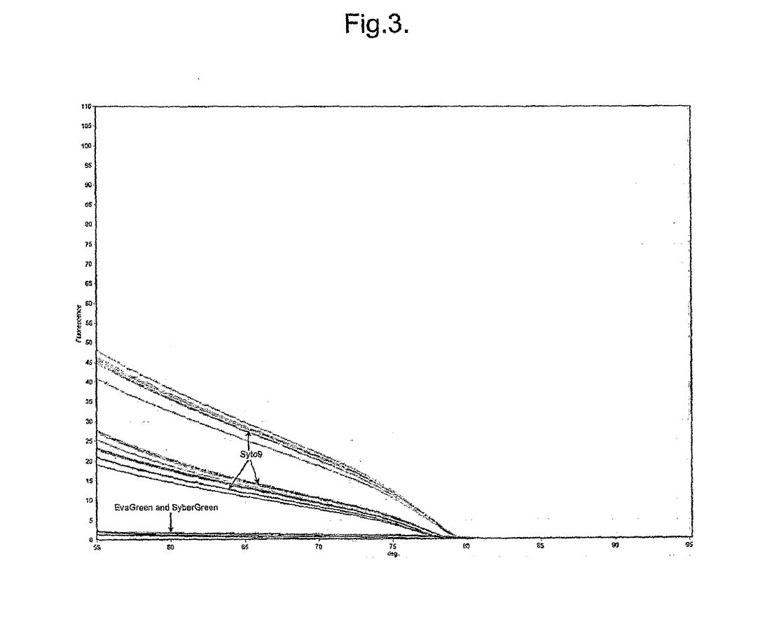

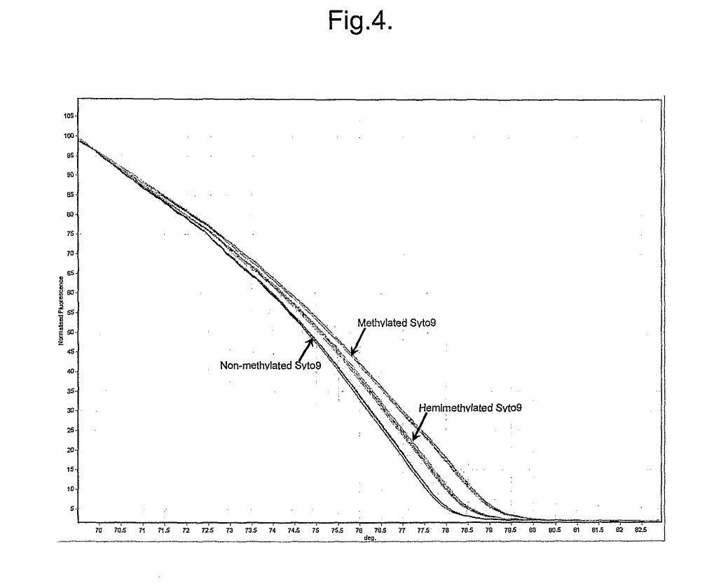

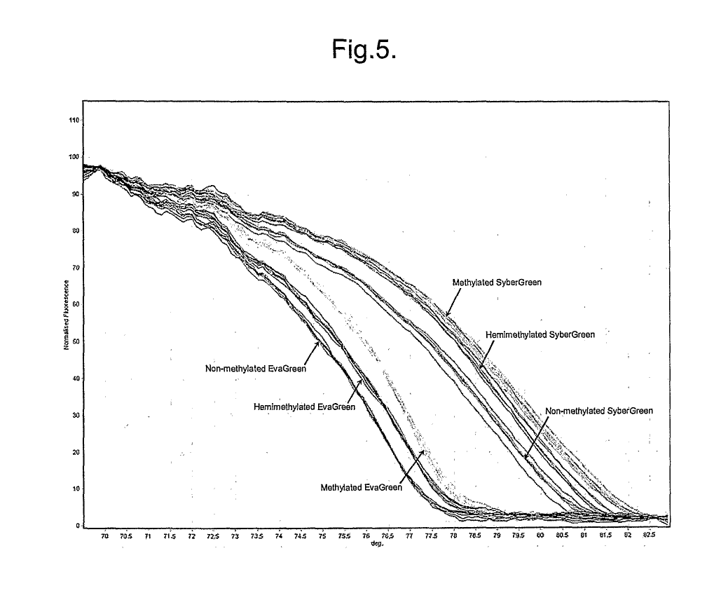

To illustrate, differences in sensitivity of methylation detection can be revealed by three commonly used dyes when exposed to identical mixed of synthesised methylated, hemi-methylated and unmethylated DNA representing the section of the CNR gene from tomato referred to previously. Here, Syto 09 showed a markedly stronger signal prior to HRM than Syber green or Eva Green (FIG. 3), and although all dyes could differentiate between the methylation status of the DNA families (FIGS. 4 and 5)

Syber green showed the clearest separation and reproducibility across replicates, followed by Eva green and then Syber green. Thus, appropriate dye selection can improve detection and sensitivity of methylated DNA. The same also applies to the now established use of the many quenching chemicals in combination with fluorescent intercalating DNA dyes.

### Amplification of Methylated DNA

A second strategy to increase sensitivity of HRM detection of methylated DNA would be to increase the concentration of the target DNA region relative to non-target DNA. Polymerase Chain Reaction (PCR) is used for this purpose in most molecular applications but is inappropriate for the detection of methylated DNA because the methylation status of template DNA is lost during the amplification steps of PCR (generating unmethylated product). This problem can be overcome if methylation status of the template strand is replicated on the newly synthesised DNA strand. This can be achieved through the use of methyl transferase enzymes.

To illustrate, we again used in vitro synthesised template DNA representing the CNR fragment (see above) and created three sample populations: unmethylated template; hemimethylated template (methylated on one strand only) and methylated template (methylated CG island on both strands). We then added two further samples in which methyl transferase was added to the hemi-methylated and methylated samples. All samples were then subjected to HRM.

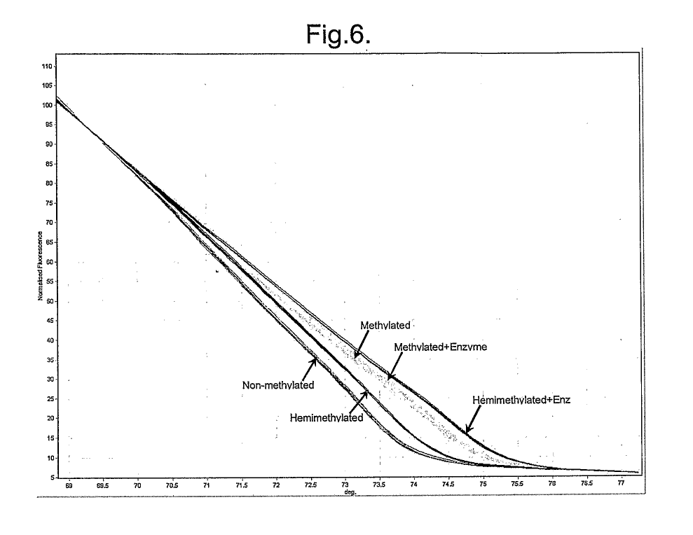

As previously noted, there was clear distinction between methylated, hemimethylated and unmethylated samples, with the methylated sample being most stable and melting at highest temperature, followed by the hemimethylated and then unmethylated samples (FIG. 6). However, when the methyl transferase was added to the hemimethylated sample, the melt transformed to become more heat stable and closely matched the methylated sample and the methylated sample treated with methyl transferase (FIG. 6). This result illustrates that the use of methyl transferase can transform HRM status of a hemimethylated DNA to become that associated with methylated DNA.

Thus, application of this treatment at each stage of a DNA amplification system such as by PCR would allow HRM-detectable signal to be preserved in the amplification products.

### REFERENCES

Finnegan E J, Genger R K, Peacock W J, Dennis E S (1998) Molecular Biology 49: 223-247.
Kainmueller, Eva K., Bannwarth, Willi (2006) HELVETICA CHIMICA ACTA 89 (12): 3056-3070
Suzuki S, Ono R, Narita T, Pask A J, Shaw G, Wang C, Kohda T, Alsop A E, Graves J A M, Kohara Y, Ishino F, Renfree M B, Kaneko-Ishino T. (2007) PLOS Genetics 3 (4): Art No. e55
Gupta S, Pathak R U, Kanungo M S (2006) Gene 377: 159-168
delGaudio R, DiGiaimo R, Geraci G (1997) FEBS Letters 417 (1):48-52
Jacobsen S E, Meyerowitz E M (1997) Science 277 (5329): 1100-1103
Melquist S, Luff B, Bender J (1999) Genetics 153 (1): 401-413
Jacobsen S E, Sakai H, Finnegan E J, Cao X F, Meyerowitz E M (2000) Current Biology 10 (4): 179-186
Cubas P, Vincent C, Coen E. (1999) Nature 401: 157-161.
Kinoshita T, Miura A, Choi Y H, Kinoshita Y, Cao X F, Jacobsen S E, Fisher R L, Kakutani T (2004) Science 303 (5657): 521-523
Finnegan E J, Peacock W J, Dennis E S (2000) Current Opinion in Genetics & Development 10 (2): 217-223
Chandler, V. L. William B. Eggleston and Jane E. Dorweiler (2000) Paramutation in maize. Plant Moecular. Biology 43, 121-145
Stokes, T. L. Barbara N. Kunkel, and Eric J. Richards. (2002) Epigenetic variation in Arabidopsis disease resistance. Genes Dev. 16, 171-182
Kakutani, T. (2002) Epi-alleles in plants: inheritance of epigenetic information over generations. Plant Cell Physiol. 43, 1106-1111
Manning Kenneth, Mahmut Tör, Mervin Poole, Yiguo Hong, Andrew J Thompson, Graham J King, James J Giovannoni & Graham B Seymour (2006) Nature Genetics 38 (8) 948-952
Monis P T, Giglio S, Saint C P. (2005) Analytical Biochemistry. May 1; 340 (1): 24-34.
Beaucage S L (2001) Current Medicinal Chemistry 8, 1213-1244
Sleutels F, Barlow D P, Lyle R. (2000) Current Opinion in Genetics and Development 10: 229-233
Bird A. (2002) Genes and Development 16: 6-21
Joyce S M, Cassells A C, Jain S M. (2003) Plant Cell, Tissue and Organ Culture 74: 103-121 Behe M. and Felsenfield G. (1981) Proceedings of the National Academy of Science U.S.A. 78: 1619-1623
Yang-Gyun Kim, Hyun-Ju Park, Kyeong Kyu Kim, Ky Lowenhaupt, and Alexander Rich. (2006) Nucleic Acids Research, 34 (17) 4937-4942

1. A method for detecting methylation of nucleic acid in a sample by
i) contacting said nucleic acid with a chemical agent that is capable of generating a detectable signal when contacted therewith;
ii) denaturing the modified nucleic acid formed in i) to a single stranded state in a steadily controlled manner;
iii) monitoring changes in detectable signal from the chemical agent in contact with nucleic acid during the denaturation step ii); and
iv) determining the level of methylation of nucleic acid by comparing the signal profile of test nucleic acid with that generated from control samples of nucleic acid having a known methylation pattern.

2. A method according to claim 1 wherein the chemical agent is selected from a fluorophore, a chromophore dye, or a chromophore dye pair suitable for use in FRET analysis.

3. A method according to claim 1 wherein the said chemical agent is capable of intercalating with the nucleic acid of the test sample.

4. A method according to claim 3 wherein the chemical agent is a fluorophore.

5. A method according to claim 4 wherein the fluorophore is a fluorescent dye.

6. A method according to claim 2 wherein the agent is a fluorophore, the method optionally comprising the step of contacting the nucleic acid with a fluorophore quenching agent.

7. A method according to claim 6 wherein the fluorescent dye is selected from the group ethidium bromide, LC Green, SYBR Green I, YO-PRO-1, BEBO and SYTO9.

8. A method according to claim 7 wherein the fluorescent dye is SYTO9.

9. A method according to claim 1 wherein the nucleic acid sample is obtained from a eukaryotic organism or a prokaryotic organism.

10. A method according to claim 9 wherein the nucleic acid sample is obtained from a eukaryotic organism.

11. A method according to claim 1 wherein the nucleic acid sample is selected from the group consisting of a synthetic DNA, a cDNA, and a chemically synthesized oligonucleotide sequence.

12. A method according to claim 1 wherein the nucleic acid sample is selected from the group consisting of genomic DNA, mitochondrial DNA, plastid DNA and cDNA.

13. A method according to claim 12 wherein the nucleic acid sample is genomic DNA.

14. A method according to claim 1 wherein the denaturing of the nucleic acid is effected by altering the stringency condition in incremental steps by a pre-determined amount in each step.

15. A method according to claim 14 wherein the stringency condition is increased.

16. A method according to claim 15 wherein the stringency condition that is incrementally increased is selected from a change in temperature or a change in chemical denaturant concentration.

17. A method according to claim 16 wherein the stringency condition that is increased is temperature over a temperature range within the range 30-90° C. at incremental steps no greater that 0.5° C.

18. A method according to claim 17 wherein the temperature range is from 40-90° C.

19. A method according to claim 17 wherein the temperature range is from 60-90° C.

20. A method according to claim 16 wherein the chemical denaturant is selected from a salt solution, a surfactant, and urea.

21. A method according to claim 20 wherein the chemical denaturant is selected from MgCl₂ or SSC.

22. A method according to claim 1 further comprising the step of amplifying the nucleic acid.

23. A method according to claim 22 whereby the amplification is by a thermocyclic enzyme amplification of DNA.

24. A method according to claim 23 whereby the amplification is conducted using the Polymerase Chain Reaction.

25. A method according to claim 22 further comprising the step of contacting the nucleic acid to a methyltransferase enzyme.

26. A method according to claim 25 wherein the nucleic acid being contacted by the methyltransferase enzyme is an amplified nucleic acid.

27-30. (canceled)

30. A kit for detecting methylation of nucleic in a sample as claimed in claim 1, said kit comprising:
(a) a chemical agent which optionally intercalates with the nucleic acid of the test sample and is selected from the group consisting of a fluorophore, a chromophore dye, or a chromophore dye pair suitable for use in FRET analysis;
(b) at least one control nucleic acid sample of known methylation status; and optionally
(c) a chemical denaturant selected from the group consisting of a salt solution, a surfactant, urea, MgCl2 or SSC.

31. A kit according to claim 30 wherein the chemical agent is capable of intercalating with the nucleic acid of the test sample.

32. A kit according to claim 30 wherein the chemical agent is a fluorescent dye selected from the group ethidium bromide, LC Green, SYBR Green I, YO-PRO-1, BEBO and SYTO9.

33. A kit according to claim 32 wherein the chemical agent is SYT09.

34. (canceled)

35. A method of improving the detection of methylation of a nucleic acid sample comprising contacting the nucleic acid sample with a methyltransferase enzyme.

36. A method as claimed in claim 35 wherein the nucleic acid sample has been amplified prior to contacting with the methyl transferase enzyme.
